(19) United States
(12) Patent Application Publication
(10) Pub. No.: US 2007/0269440 A1
(43) Pub. Date: Nov. 22, 2007

(54) Stimulators of osteoblastogenesis and applications of the same

(75) Inventor: Charles K. Lumpkin, Little Rock, AR (US)

Correspondence Address: L. Joseph Underwood;Williams & Anderson PLC, Twenty-Second Floor, 111 Center St., Little Rock, AR, 72201, US

(73) Assignee: Board of Trustees of the University of Arkansas, Little Rock, AR (US)

(21) Appl. No.: 11/439,294

(22) Filed: May 22, 2006

(51) Int. Cl. A61K 39/395; A61K 38/17

(52) U.S. Cl. 424/145.1; 514/12

(57) ABSTRACT

Compositions that stimulate osteoblastogenesis and the activity of osteoblasts are disclosed. Methods of stimulating osteoblastogenesis and the activity of osteoblasts are disclosed. Methods of treating a mammal for a bone condition by stimulating osteoblastogenesis and the activity of osteoblasts are disclosed. According to the present invention, a composition comprising a TNF blocker is administered to the mammal in an amount effective to stimulate osteoblastogenesis.

### STATEMENT REGARDING FEDERALLY SPONSORED RESEARCH

Development of this invention was supported by Grant 5R01-AA012223-06 from the National Institutes of Health. The United States government may have certain rights in this invention.

### BACKGROUND OF THE INVENTION

Tumor necrosis factor (TNF) is primarily a product of the monocyte-macrophage lineage of cells and mediates proliferation, inhibition, differentiation, and activation of a variety of cell types (1). Typical cells that synthesize TNF are, for example, bone marrow stromal cells, T cells, and osteoblasts.

TNF refers to two proteins, TNF-α and TNF-β. These two proteins have approximately 30% amino acid identity and share receptors. TNF interacts with two receptors, TNF receptor Type I (TNF-RI) and TNF receptor Type II (TNF-RII), collectively TNF-R. TNF antagonists may be found in soluble forms, which are derived from the extracellular domains of TNF-RI or TNF-RII. Soluble TNF-R (sTNF-R) may mean, for example, the extracellular domain of the human TNF-R1 linked to a molecule of polyethylene glycol (2).

Distraction osteogenesis (DO) is a unique clinical method of bone formation and can be considered a type of fracture healing that stretches the biological repair process to its natural limits. Ilizarov first developed this method both experimentally and clinically over his forty-year career in Siberia (3). DO results from slowly-pulling apart the edges of a bone fracture or osteotomy, with an external fixator, to permit rapid formation of new bone in the slowly expanding gap.

The DO process may be divided into two major phases. The first phase is the distraction phase in which the edges of the fracture or osteotomy are stretched apart and in which processes of direct bone formation (intramembranous, appositional, osteoblastogenesis) occur in the gap. This is followed by the consolidation phase, beginning after the stretching stops, where bone bridging and the osteoclast-mediated processes of bone resorption and remodeling are initiated at each host bone surface to reform the medullary canal.

Following approximately 7 days of distraction in a mammal, such as, for example, a human, a mouse or a rat, the distraction gap is bridged by five biologically active, spatially oriented zones between the fracture sites. The central zone, termed the fibrous interzone (FIZ), contains mesenchymal cells associated with parallel collagen bundles. The FIZ is bounded on both sides by the primary matrix front (PMF), where mesenchymal stem cells proliferate and differentiate into osteoblasts and bone matrix deposition occurs. The PMF zones are, in turn, bounded on both sides by the zones of microcolumn formation (MCF) where bone matrix expands around osteoblasts or osteocytes into parallel microcolumns. Thus, DO can be used to test for the effects of various factors on osteoblastogenesis. To date, the above discussion of DO is equally applicable to many mammals, such as, for example, humans, rats, and mice (4, 5).

Many bone pathologies undergo bone formation and bone resorption processes in a manner that has similarities to the process described for distraction osteogenesis. One example of such is a bone fracture. A fracture may be simple or compound. The point of fracture heals through the regenerative process of osteoblastogenesis. Compositions of the present invention may be administered to a mammal with a fracture thereby increasing or stimulating osteoblastogenesis and healing.

Similarly, the process of osteoblastogenesis is necessary in many orthopedic procedures, such as, for example, implantation of a prosthesis. Osteoblastogenesis forms new bone around a prosthetic device and, in some cases, into a prosthetic device, thereby anchoring the prosthesis firmly in the bone. Compositions of the present invention may be administered to a recipient of a prosthesis thereby stimulating osteoblastogenesis and increasing osseointegration of said prosthesis. In some instances, a prosthesis, such as, for example, a prosthesis used in an artificial joint, may loosen with use. Compositions of the present invention may be administered to a patient whose prosthesis has loosened, thereby stimulating osteoblastogenesis and osteoblast activity for osseointegration of the loosened prosthesis.

Additionally, osteoarthritis is an inflammatory disease that negatively affects the joints and bone surfaces of joints. TNF is a pro-inflammatory cytokine. Administration of TNF blockers such as, for example, the compositions disclosed in the present invention, limit the action of TNF, thereby decreasing the inflammation associated with osteoarthritis and decreasing the concomitant bone conditions associated with osteoarthritis.

### SUMMARY OF THE INVENTION

The present invention relates to compositions that stimulate osteoblastogenesis and the activity of osteoblasts. The present invention also relates to methods of stimulating osteoblastogenesis and the activity of osteoblasts. Furthermore, methods of treating a mammal suffering from a bone condition by stimulating osteoblastogenesis and the activity of osteoblasts are disclosed. According to the present invention, a composition comprising a TNF blocker is administered to a mammal with a bone condition in an amount effective to stimulate osteoblastogenesis.

### BRIEF DESCRIPTION OF THE DRAWINGS

FIG. 1 illustrates a schematic depicting the stainless steel external fixator including pin placement and orientation of the tibia used in osteodistraction of mice tibia. Note that the isometric view is drawn with the distal end of the tibia at the top.

FIG. 2 illustrates endosteal bone formation during distraction osteogenesis in young and old mice by single-beam radiography and histology. Strict inclusion criteria limited analysis to n=5 per group for radiography, n=8 for young histology, and n=6 for old histology. Mineralization of the DO gap, as measured from the radiographs, was measured as 51.4±5.4% SEM of the distraction gap in 4-month old and 33.5±4.8% SEM in 12-month old mice (^{a}p<0.039). Analysis of histological sections revealed a similar, yet more significant, trend in the formation of new bone columns (young=57.1±3.7% SEM; old=25.1±6.5% SEM; ^{b}p<0.001).

FIG. 3 illustrates representative histological sections of distracted tibiae from 4-month-old (A) and 12-month-old (B) mice demonstrating an age-related deficit in endosteal bone formation after a 6-day latency and 14 days of distraction. The endosteal new bone has been roughly outlined for demonstration purposes only. Qualitatively, the newly formed bone was oriented along the tension vector. C, cortex; NB, new bone. Original magnifications 4×.

FIG. 4 illustrates a representative histological section of a distracted tibia from an aged mouse demonstrating an age related deficit of endosteal bone formation after a 3-day latency and 14 days of distraction.

FIG. 5 illustrates a representative histological section of a distracted tibia from an aged mouse demonstrating an age related deficit of endosteal bone formation after a 3-day latency and 14 days of distraction.

FIG. 6 illustrates a representative histological section of a distracted tibia from an aged mouse demonstrating an age related deficit of endosteal bone formation after a 3-day latency and 14 days of distraction.

FIG. 7 illustrates a representative histological section of a distracted tibia from an aged mouse treated with sTNF-RI demonstrating endosteal bone formation after a 3-day latency and 14 days of distraction.

FIG. 8 illustrates a representative histological section of a distracted tibia from an aged mouse treated with sTNF-RI demonstrating endosteal bone formation after a 3-day latency and 14 days of distraction.

FIG. 9 illustrates a representative histological section of a distracted tibia from an aged mouse treated with sTNF-RI demonstrating endosteal bone formation after a 3-day latency and 14 days of distraction.

FIG. 10 illustrates endosteal bone formation during distraction osteogenesis in old mice in the presence or absence of sTNF-RI as measured by single-beam radiography. Mineralization of the DO gap, as measured from the radiographs, was calculated as 27.3±8.4% SEM of the distraction gap in 21-month old control mice and 55.2±8.8% SEM in 21-month old mice treated with sTNF-RI (^{a}p<0.038).

FIG. 11 illustrates endosteal bone formation during distraction osteogenesis in old mice in the presence or absence of sTNF-RI as measured by histology. Analysis of histological sections revealed a similar trend to radiography in the formation of new bone columns. Mineralization of the DO gap, as measured histologically, was calculated as 31.2±12.4% SEM of the distraction gap in 21-month old control mice and 76.1±7.5% SEM in 21-month old mice treated with sTNF-RI (^{b}p<0.009).

### DETAILED DESCRIPTION

The present invention is particularly described in the following description and examples that are intended as being illustrative and not limiting as various modifications or substitutions will be apparent to those skilled in the art.

As used herein, the terms “stimulating” or “stimulate” refers to an increase in the amount, quality or effect of a particular activity.

As used herein, the term “TNF” may refer to either or both Tumor Necrosis Factor-alpha (TNF-α) and Tumor Necrosis Factor-beta (TNF-β).

As used herein, the term “TNF blocker” refers to a TNF antagonist. A TNF blocker inhibits the activity of TNF. Examples of TNF blockers include, but are not limited to, etanercept (ENBREL™) from Immunex Corporation, infliximab (REMICADE™) from Centocor, Inc., soluble TNF receptor Type I (sTNF-RI or sTNFRI), pegylated-sTNF-RI, soluble TNF receptor Type II (sTNF-RII or sTNF-RII), pegylated-sTNF-RII, CDP571 (a humanized monoclonal anti-TNF-alpha antibody), D2E7 (a human anti-TNF monoclonal antibody), other anti-TNF-alpha antibodies, and TNF-alpha converting enzyme inhibitors. Additionally, the said TNF blocker may be, for example, thalidomide, phosphodiesterase 4 (IV) inhibitor thalidomide analogs and other phosphodiesterase 4 (IV) inhibitors.

As used herein, the term “sTNFR” or “sTNF-R” refers to a molecule comprising a soluble TNF receptor protein, either Type I or Type II, or a fragment thereof.

As used herein, the term “bone condition” refers to any disease, condition, disorder, syndrome, or trauma of or to a bone which would be benefited, treated, rescued or healed by osteoblastogenesis. Examples of such bone conditions include, but are not limited to, fracture, delayed unions, non-unions, distraction osteogenesis, osteotomy, osseointegration, and osteoarthritis.

As used herein, the term “fracture” refers to a fracture of a skeletal bone, whether simple or compound.

As used herein, the term “distraction osteogenesis” refers to the process of lengthening bones or repairing skeletal deformities comprising increasing the size of a gap between sections of bone and allowing new bone to grow in said gap.

As used herein, the term “osteotomy” refers to a surgical sectioning or surgical drilling of bone.

As used herein, the term “osseointegration” refers to the firm anchoring of a surgical implant or prosthesis (as in dentistry or in bone surgery) by the growth of bone around or into said surgical implant or prosthesis without fibrous tissue formation at the interface of said bone and said surgical implant or prosthesis. The term osseointegration includes integration of new implants or prostheses and reversal of osteolytic loosening of implants or prostheses.

As used herein, the term “osteoarthritis” refers to the disease or condition of osteoarthritis.

As used herein, the term “therapeutically effective dosage” refers to an amount of a composition which produces a medicinal effect observed as an increase in osteoblastogenesis in a mammal when a therapeutically effective dosage of said composition is administered to said mammal having a bone condition.

As used herein, the term “osteoblastogenesis” refers to an increase in the presence and activity of osteoblasts and the direct formation of bone tissue by said osteoblasts.

The present invention provides a method of stimulating osteoblastogenesis to treat a bone condition in a mammal by administering a therapeutically effective dosage of a TNF blocker to said mammal. The said bone condition may be a bone condition such as, for example, fracture, delayed unions, non-unions, distraction osteogenesis, osteotomy, osseointegration, and osteoarthritis. The said TNF blocker may be, for example, etanercept, infliximab, soluble TNF receptor Type I, soluble TNF receptor Type II, CDP571, D2E7, other anti-TNF antibodies, TNF-alpha converting enzyme inhibitors. Additionally, said TNF blocker may be, for example, thalidomide, phosphodiesterase 4 (IV) inhibitor thalidomide analogs and other phosphodiesterase 4 (IV) inhibitors.

In one embodiment of the present invention, said TNF blocker may be administered to a mammal in a pharmaceutically acceptable carrier. Furthermore, administering a therapeutically effective dosage of a TNF blocker to said mammal may be, for example, through any of the following routes: intravenous, intramuscular, oral, subcutaneous, intrathecal, intranasal, transepidermal, parenteral, by inhalation or Azlet pump and catheter.

In one embodiment of the present invention, a method is disclosed for stimulating osteoblastogenesis to treat a bone condition in a mammal by administering a therapeutically effective dosage of a TNF blocker to said mammal. Particularly, the mammal may be, for example, a mouse or a human.

In particular embodiments of the present invention, the therapeutically effective dosage of said TNF blocker may be from about 1 mg/kg/day, to about 2 mg/kg/day, to about 4 mg/kg/day, to about 6 mg/kg/day, to about 8 mg/kg/day, to about 10 mg/kg/day, to about 15 mg/kg/day, to about 20 mg/kg/day, to about 25 mg/kg/day, or to about 50 mg/kg/day.

In particular embodiments of the present invention, the therapeutically effective dosage of said TNF blocker may be from about 1 mg/kg, to about 2 mg/kg, to about 4 mg/kg, to about 6 mg/kg, to about 8 mg/kg, to about 10 mg/kg, to about 15 mg/kg, to about 20 mg/kg, to about 25 mg/kg, or to about 50 mg/kg.

In particular embodiments of the invention, the therapeutically effective dosage of said TNF blocker may be administered subcutaneously in said mammal and said dosage may be in the range of 5 mg to about 10 mg, to about 20 mg, to about 30 mg, to about 40 mg or to about 50 mg.

In particular embodiments of the invention, the therapeutically effective dosage of said TNF blocker may be administered intranasally in said mammal and said dosage may be in the range of 0.1 mg to about 0.5 mg, to about 1 mg, to about 3 mg, to about 5 mg, to about 8 mg, or to about 10 mg.

In particular embodiments of the invention, the therapeutically effective dosage of said TNF blocker may be administered transepidermally in said mammal and said dosage may be in the range of 10 mg to about 20 mg, to about 30 mg, to about 40 mg, to about 50 mg, to about 60 mg, to about 70 mg, to about 80 mg, to about 90 mg, or to about 100 mg.

In particular embodiments of the invention, the therapeutically effective dosage of said TNF blocker may be administered by inhalation in said mammal and said dosage may be in the range of 0.2 mg to about 0.5 mg, to about 1.0 mg, to about 5 mg, to about 10 mg, to about 20 mg, to about 30 mg, or to about 40 mg.

In particular embodiments of the invention, the therapeutically effective dosage of said TNF blocker may be administered intravenously in said mammal and said dosage may be a therapeutically effective amount.

In particular embodiments of the invention, the therapeutically effective dosage of said TNF blocker may be administered orally in said mammal and said dosage may be in the range of 10 mg to about 20 mg, to about 30 mg, to about 40 mg, to about 50 mg, to about 100 mg, to about 150 mg, to about, 200 mg, to about 250 mg, or to about 300 mg.

In one embodiment of the present invention, said TNF blocker may be etanercept and the therapeutically effective dosage may be administered intramuscularly in said mammal and said dosage may be in the range of 25 mg, to about 30 mg, to about 50 mg, to about 80 mg, or to about 100 mg. In a particular embodiment, said TNF blocker may be etanercept and the therapeutically effective dosage may be 30 mg. In a particular embodiment, said TNF blocker may be etanercept and the therapeutically effective dosage may be 60 mg. In a particular embodiment, said TNF blocker may be etanercept and the therapeutically effective dosage may be 90 mg.

In one embodiment of the present invention, said TNF blocker may be etanercept and the therapeutically effective dosage may be administered subcutaneously in said mammal and said dosage may be in the range of 5 mg, to about 10 mg, to about 20 mg, to about 30 mg, to about 40 mg, or to about 50 mg.

In one embodiment of the present invention, said TNF blocker may be etanercept and the therapeutically effective dosage may be administered intrathecally in said mammal and said dosage may be in the range of 0.1 mg, to about 0.5 mg, to about 1.0 mg, to about 5 mg, to about 10 mg, to about 15 mg, to about 20 mg, or to about 25 mg administered once a day, once a week, once every two weeks, once every month, once every two months or once every three months.

In one embodiment of the present invention, said TNF blocker may be infliximab and the therapeutically effective dosage may be administered intravenously in said mammal and said dosage may be in the range of 2.5 mg/kg to about 5 mg/kg, to about 10 mg/kg, to about 15 mg/kg, or to about 20 mg/kg.

In one embodiment of the present invention, said TNF blocker may be infliximab and the therapeutically effective dosage may be administered intrathecally in said mammal and said dosage may be in the range of 0.1 mg/kg to about 0.5 mg/kg, to about 1 mg/kg, to about 2 mg/kg, to about 3 mg/kg, to about 4 mg/kg or to about 5 mg/kg administered once a day; once a week, once every two weeks, once every month, once every two months or once every three months.

Another embodiment of the present invention provides for a pharmaceutical composition comprising a TNF blocker in an amount effective to stimulate osteoblastogenesis for treating a bone condition in a mammal.

In particular embodiments, the pharmaceutical composition is used to treat a bone condition selected from the group consisting of fracture, delayed unions, non-unions, distraction osteogenesis, osteotomy, osseointegration, and osteoarthritis.

In particular embodiments, the pharmaceutical composition comprises a TNF blocker selected from the group consisting of etanercept, infliximab, soluble TNF receptor Type I, soluble TNF receptor Type II, CDP571, and D2E7.

In particular embodiments, the pharmaceutical composition is used to treat a mammal wherein said mammal is a human.

Further embodiments of the present invention provide a method of stimulating osseointegration of a prosthesis in a mammal comprising administering a therapeutically effective dosage of a TNF blocker to said mammal.

In particular embodiments, the prosthesis comprises a dental prosthesis.

In particular embodiments, the prosthesis comprises a prosthetic joint or a part of a prosthetic joint.

In particular embodiments, the prosthesis comprises a prosthetic bone.

In particular embodiments, the prosthesis is implanted in a mammal wherein said mammal is a human.

In particular embodiments, the TNF blocker is applied to the prosthesis prior to implantation of the prosthesis.

In further embodiments, the TNF blocker may be administered directly to the implantation site of a prosthesis.

### EXAMPLES

The following examples are further illustrative of the present invention, but it is understood that the invention is not limited thereto.

### Example 1

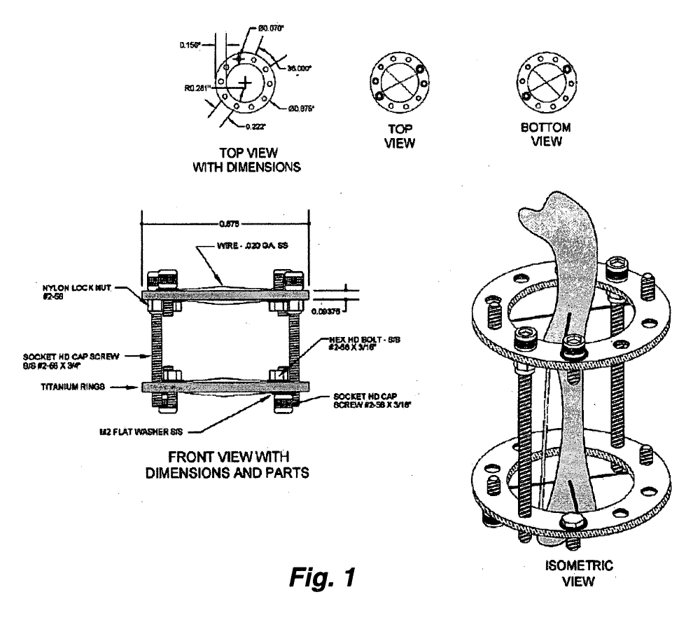

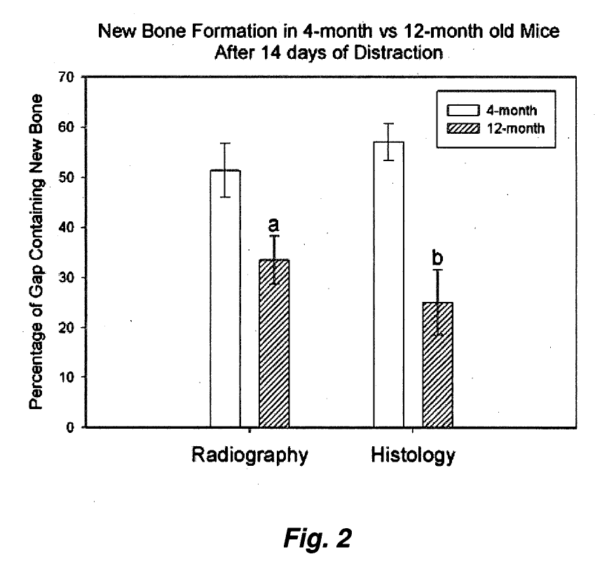

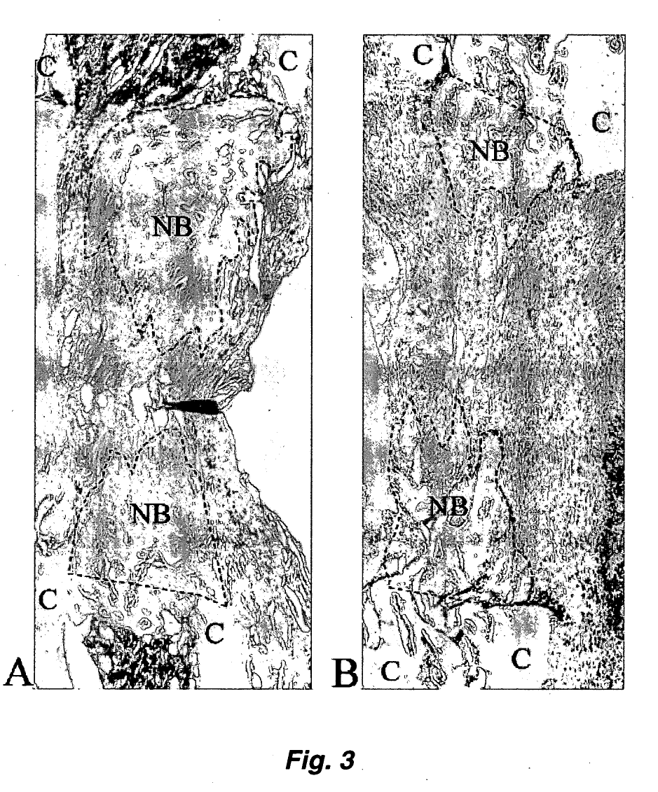

This study was conducted to demonstrate that aging in mice results in decreased bone formation during DO. Four-month old and twelve-month old CB57BL/6 male mice (n=10 per group) underwent DO. External fixators were placed on the left tibiae and mid-diaphyseal tibial osteotomies were performed immediately following fixator placement (See FIG. 1). Distraction, which began six days after surgery at 0.075 mm b.i.d. (0.15 mm/day) for 14 days, resulted in a total lengthening of 2.1 mm. Following distraction, the distracted tibiae were removed for high-resolution radiography and histological evaluation. Radiographic analysis demonstrated a significant decrease in the mineralized area of distraction gaps of 12-month (33.5%±4.8) versus 4-month (51.4%±5.4) mice (P<0.039). Histological analysis of representative specimens confirmed the decrease in bone formation observed in the radiographs (P<0.001). Endosteal new bone was predominantly intramembranous and appeared highly oriented toward the distraction axis (See FIG. 2 and FIG. 3). These results demonstrate that 12-month old mice have a relative deficit in endosteal bone formation compared to that in younger mice.

For initial radiography a Xerox Micro 50 closed system radiography unit (Xerox, Pasadena, Calif.) was used at 40 kilovolts (3 mA) for 20 seconds with Kodak X-OMAT film to radiograph the specimens. For quantitation, the radiographs and histology slides are video recorded by radio camera under low power (1.25× objective) microscopic magnification and transferred into a computer (Macintosh IIx Image 1.28c). The radiographs allow for quantitation of mineralized bone within the distraction gap. The measured distraction gap area was outlined from the outside corners of the two proximal and the two distal cortices forming a quadrilateral region of interest. The mineralized new bone area in the gap was determined by outlining the regions with radiodensity equivalent to or greater than the adjacent medullary bone. Mean pixel density of the defined distraction gap was measured using NIH Image on an arbitrary 256 degree scale. Gaps were measured to ensure that all were of comparable length. The percentage of new mineralized bone area within the distraction gap (% new bone) was calculated by dividing mineralized bone area/gap area. (6, 7).

After radiography, the distracted tibiae were decalcified in 5% formic acid. The specimens were paraffin-embedded and cut into 5-7 micron longitudinal (coronal) sections on a microtome (Leitz 1512, Wetzlar, Germany) for staining (hematoxylin & eosin). The sections chosen for analysis were selected to represent a central or near central gap location. This was accomplished by choosing slides that contained all four full thickness cortices with intact marrow spaces on both the proximal and distal ends of the distraction gap. All organized osteoid/sinusoid columns were defined as new bone. We defined periosteal new bone as that outside of each cortex and endosteal new bone as that within the cortices. In selected specimens, progressive sections were taken end to end from one peripheral cortex to the opposite side cortex in 50 micron increments to judge the reproducibility of the mid-coronal sampling technique. The distraction gap area was outlined from the outside corners of the two proximal and the two distal cortices forming a quadrilateral region of interest, and the area of that region (gap area) was recorded. Both the proximal and distal endosteal new bone matrix, which is easily distinguished from the central fibrous tissue at the PMF, was outlined and the area recorded (new bone area). The percentage of new bone within the distraction gap was calculated by dividing new bone area/gap area (8, 9-11).

The H&E staining allows calculation of the relative percentages of matrix or cell types within specific zones in the gap. Alternatively, the toluidine blue stain allows for identification and quantitation of cartilage (endochondral bone) formation (data not shown). Additionally, the Masson trichrome stain allows for identification of premineralized matrix (osteoid) (data not shown).

### Example 2

Twenty-four 21-month-old C57BL6 mice underwent placement of an external fixator and osteotomy to the left tibia. The surgeries were completed over a four-day period. After surgeries there were eleven mice in the control group (one died during surgery) and twelve mice in the group to be treated with sTNF-RI. Distraction began three days after surgery (three day latency) at a rate of 0.075 mm b.i.d and continued for 14 days. On the first day of distraction all mice received a subcutaneous injection of either vehicle (PBS pH 7.4) for control mice or sTNF-RI (8.0 mg/kg) in vehicle for the query group. The injections were administered every other day for the remainder of the study. At sacrifice, the distracted and contra lateral tibiae were harvested and trunk blood was collected for serum. Radiologic and histologic analyses were performed as previously described.

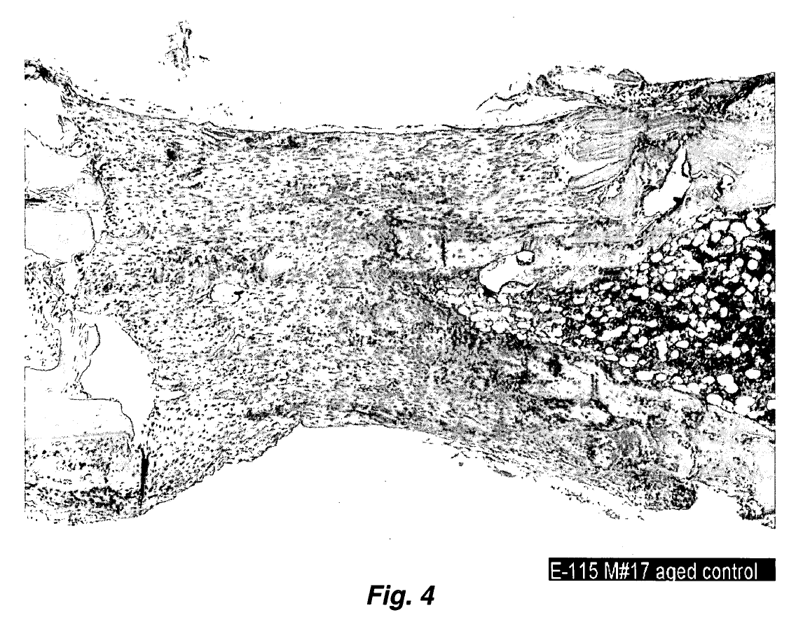

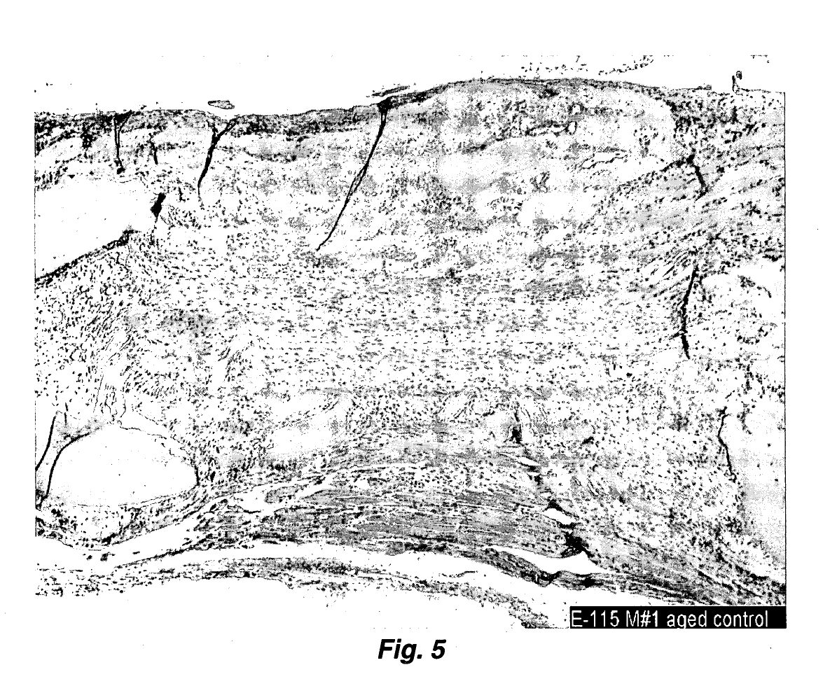

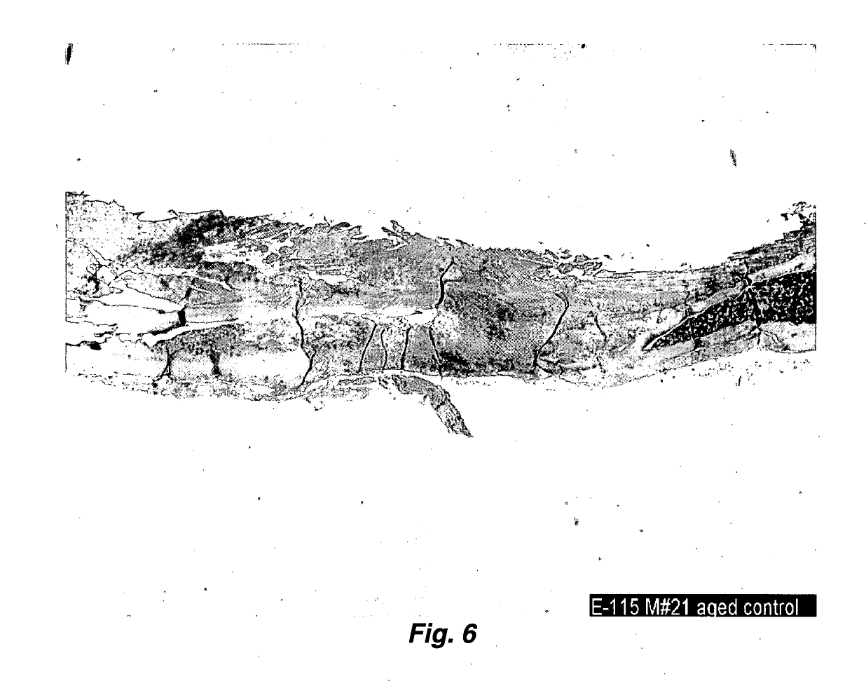

FIGS. 4-6 illustrate samples of distracted tibiae from three individual aged mice that underwent osteotomy and distraction osteogenesis and treatment with vehicle alone as controls.

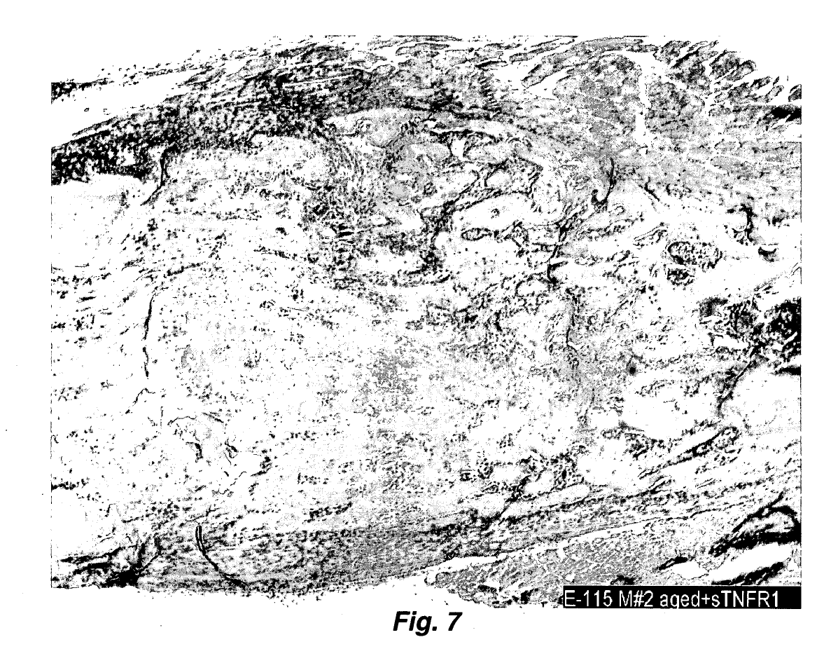

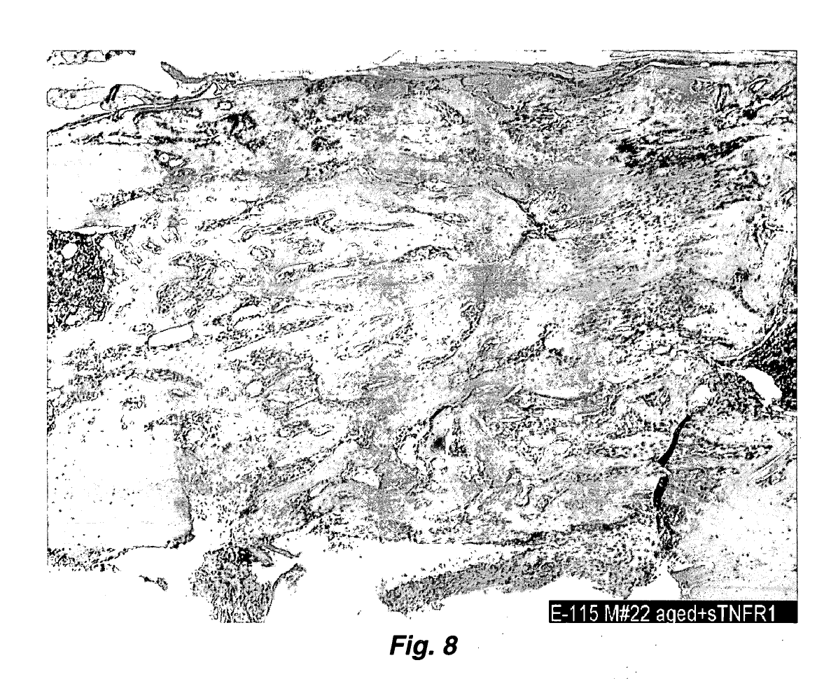

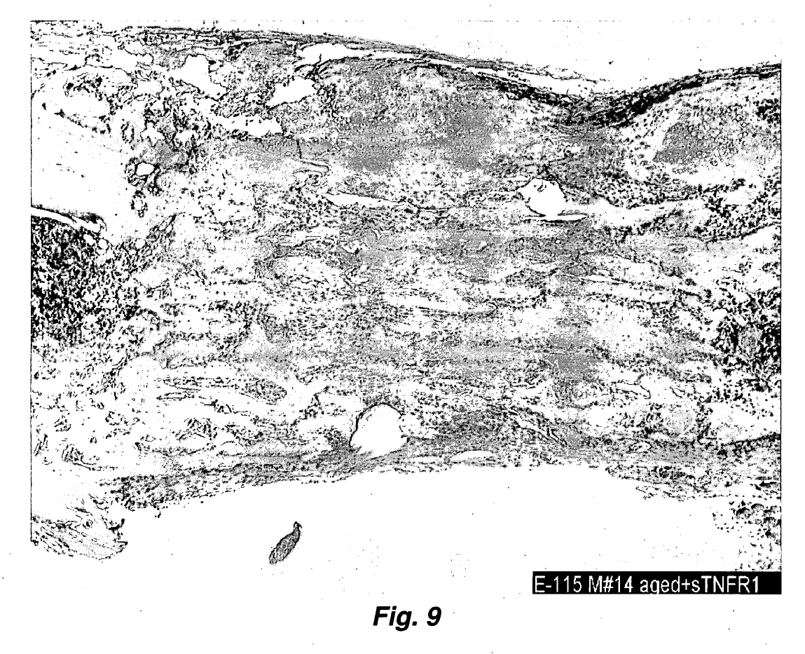

FIGS. 7-9 illustrate samples of distracted tibiae from three individual aged mice that underwent osteotomy and distraction osteogenesis and treatment with sTNF-RI as described above.

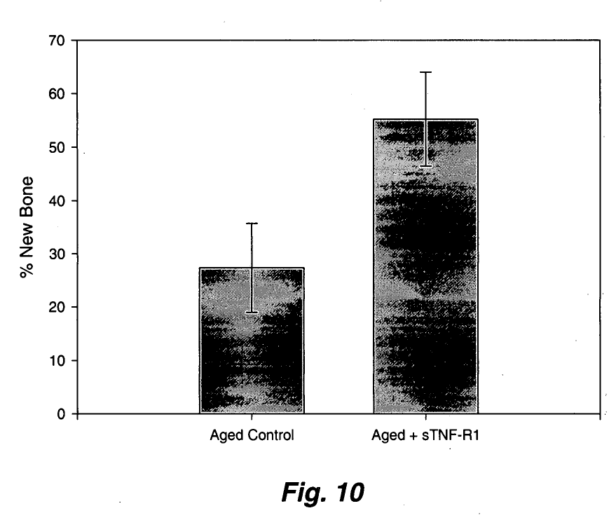

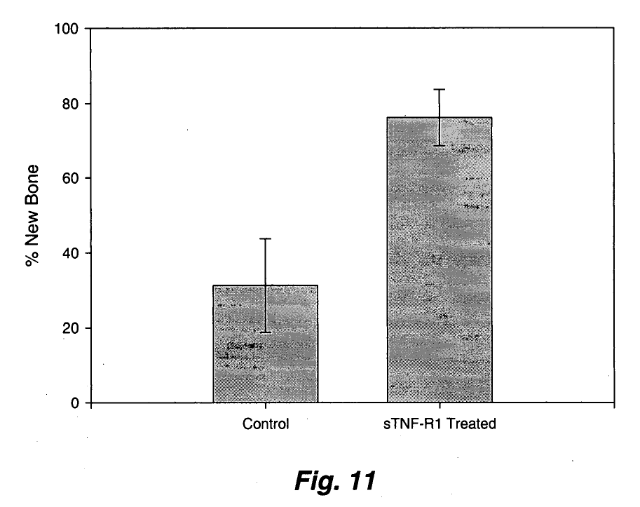

FIGS. 10 and 11 graphically illustrate the percentage of new bone formed for both the control group and the sTNF-RI treated mice as measured radiologically (FIG. 10) and histologically (FIG. 11).

These data clearly indicate that TNF blockers stimulate osteoblastogenesis and new bone formation.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinence of the cited references.

As various changes could be made in the above methods and compositions without departing from the scope of the invention, it is intended that all matter contained in the above description be interpreted as illustrative and not in a limiting sense. Unless explicitly stated to recite activities that have been done (i.e., using the past tense), illustrations and examples are not intended to be a representation that given embodiments of this invention have, or have not, been performed.

### REFERENCES

1. Assuma R, Oates T, Cochran D, Amar S, Graves D T. IL-1 And TNF Antagonists Inhibit Inflammatory Response And Bone Loss In Experimental Periodontitis. J Immunology 160:403-409, 1998.
2. Kimble R B, Srivastava S, Ross F P, Matayoshi A, Pacifici R. Estrogen Deficiency Increases The Ability Of Stromal Cells To Support Murine Osteoclastogenesis Via An Interleukin-1 And Tumor Necrosis Factor-Mediated Stimulation Macrophage Colony Stimulating Factor Production. J Biol Chem 271:28890-28897, 1996.
3. Ilizarov Ga. Clinical Application Of The Tension-Stress Effect For Limb Lengthening. Clin Orthop Rel Res, 250: 8, 1990.
4. Aronson J. Experimental And Clinical Experience With Distraction Osteogenesis. Cleft Palate-Craniofacial J, 31:473-482, 1994.
5. Tay B, Le A, Gould S, Helms J. Histochemical And Molecular Analyses Of Distraction Osteogenesis In A Mouse Model. J Orthop Res 16:636-642, 1998.
6. Aronson J, Gao G, Shen X, Mclaren S, Skinner R, Badger T, Lumpkin C. The Effect Of Aging On Distraction Osteogenesis In The Rat. J Ortho Res 19:421-427, 2001.
7. Skinner R A, Fromowitz F B. Faxitron X-ray Unit in Histologic Evaluation of Breast Biopsies. Tech Sample HT-5. ASCP Press. Chicago, Ill., 1989.
8. Brown E, Perrien D, Fletcher T, Irby D, Aronson J, Gao G, Hogue W, Skinner R, Suva L, Ronis M, Hakkak R, Badger T, Lumpkin C. Skeletal Toxicity Associated With Chronic Ethanol Exposure In A Rat Model Using Total Enteral Nutrition. J Pharm Exper Therap 301:1132-1138, 2002.
9. Skinner R A, Nicholas R. Preparing Superior Quality Slides of Orthopedic Tissue Using Previously Decalcified Material. Tech Sample HT 2. ASCP Press. Chicago, Ill., 1990.
10. Skinner R A, Nicholas R W, Stewart C L, Vireday C. Resected Allograft Bone Containing an Implanted Expanded Polytetrafluoroethylene Prosthetic Ligament: Comparison of Paraffin, Glycolmethacrylate, and Exakt Grinding Techniques. J Histotech 16:129-137, 1993.
11. Skinner R A, Hickmon S G, Lumpkin C K Jr, Aronson J, Nicholas R W. Decalcified Bone: Twenty Years of Successful Specimen Management. J Histotech 20:267-277, 1997.

What is claimed is:

1. A method of stimulating osteoblastogenesis to treat a bone condition in a mammal comprising administering a therapeutically effective dosage of a TNF blocker to said mammal.

2. The method of claim 1, wherein said bone condition is selected from the group consisting of fracture, delayed unions, non-unions, distraction osteogenesis, osteotomy, osseointegration, and osteoarthritis.

3. The method of claim 1, wherein said TNF blocker is selected from the group consisting of etanercept, infliximab, soluble TNF receptor Type I, soluble TNF receptor Type II, CDP571, and D2E7.

4. The method of claim 1, wherein said TNF blocker is administered in a pharmaceutically acceptable carrier.

5. The method of claim 1, wherein said mammal is a human.

6. The method of claim 1, wherein administering a therapeutically effective dosage of a TNF blocker to said mammal is through any of the following routes: intravenous, intramuscular, oral, subcutaneous, intrathecal, intranasal, transepidermal, parenteral, by inhalation or by Azlet pump and catheter.

7. The method of claim 1, wherein the therapeutically effective dosage of said TNF blocker is in the range of 1 mg/kg/day to 50 mg/kg/day.

8. The method of claim 1, wherein the therapeutically effective dosage of said TNF blocker is in the range of 1 mg/kg to 50 mg/kg.

9. The method of claim 1, wherein the therapeutically effective dosage of said TNF blocker is administered subcutaneously in said mammal and wherein said dosage is in the range of 5 mg to 50 mg.

10. The method of claim 1, wherein the therapeutically effective dosage of said TNF blocker is administered intranasally in said mammal and wherein said dosage is in the range of 0.1 mg to 10 mg.

11. The method of claim 1, wherein said TNF blocker is administered transepidermally in said mammal and wherein said dosage is in the range of 10 mg to 100 mg.

12. The method of claim 1, wherein said TNF blocker is administered by inhalation in said mammal and wherein said dosage is in the range of 0.2 mg to 40 mg.

13. The method of claim 1, wherein said TNF blocker is administered intravenously in said mammal and wherein said dosage is a therapeutically effective amount.

14. The method of claim 1, wherein said TNF blocker is administered orally to said mammal and wherein said dosage is in the range of 10 mg to 300 mg.

15. The method of claim 1, wherein said TNF blocker is etanercept and the therapeutically effective dosage is administered intramuscularly in said mammal and wherein said dosage is in the range of 25 mg to 100 mg.

16. The method of claim 1, wherein said TNF blocker is etanercept and the therapeutically effective dosage is administered subcutaneously in said mammal and wherein said dosage is in the range of 5 mg to 50 mg.

17. The method of claim 1, wherein said TNF blocker is etanercept and the therapeutically effective dosage is administered intrathecally in said mammal and wherein said dosage is in the range of 0.1 mg to 25 mg administered from once a day to once every three months.

18. The method of claim 1, wherein said TNF blocker is infliximab and the therapeutically effective dosage is administered intravenously in said mammal and wherein said dosage is in the range of 2.5 mg/kg to 20 mg/kg.

19. The method of claim 1, wherein said TNF blocker is infliximab and the therapeutically effective dosage is administered intrathecally in said mammal and wherein said dosage is in the range of 0.1 mg/kg to 5 mg/kg administered from once a week to once every three months.

20. A method of stimulating osseointegration of a prosthesis in a mammal comprising administering a therapeutically effective dosage of a TNF blocker to said mammal.

21. The method of claim 20, wherein said prosthesis comprises a dental prosthesis.

22. The method of claim 20, wherein said prosthesis comprises a prosthetic joint.

23. The method of claim 20, wherein said prosthesis comprises a prosthetic bone.

24. The method of claim 20, wherein said mammal is a human.

25. The method of claim 20, wherein said prosthesis comprises a TNF blocker.

26. The method of claim 20, wherein said TNF blocker is administered directly to the implantation site of the prosthesis.

27. A pharmaceutical composition comprising a TNF blocker in an amount effective to stimulate osteoblastogenesis for treating a bone condition in a mammal.

28. The pharmaceutical composition of claim 27, wherein said bone condition is selected from the group consisting of fracture, delayed unions, non-unions, distraction osteogenesis, osteotomy, osseointegration, and osteoarthritis.

29. The pharmaceutical composition of claim 27, wherein said TNF blocker is selected from the group consisting of etanercept, infliximab, soluble TNF receptor Type I, soluble TNF receptor Type II, CDP571, and D2E7.

30. The pharmaceutical composition of claim 27, wherein said mammal is a human.
